(19) United States
(12) Patent Application Publication
(10) Pub. No.: US 2021/0251786 A1
(43) Pub. Date: Aug. 19, 2021

(54) SINGLE FIBER CONSTRAINING FOR IMPLANTABLE MEDICAL DEVICES

(71) Applicant: W. L. Gore & Associates, Inc., Newark, DE (US)

(72) Inventor: Tyson J. Skelton, Flagstaff, AZ (US)

(21) Appl. No.: 17/251,365

(22) PCT Filed: Jun. 14, 2018
(86) PCT No.: PCT/US2018/037586

§ 371 Date: Dec. 11, 2020

(51) Int. Cl. A61F 2/95

(52) U.S. Cl. CPC A61F 2/95; A61F 2210/0076; A61F 2250/0067; A61F 2002/9511

(57) ABSTRACT

Various aspects of the present disclosure are directed toward apparatuses, systems, and methods that include an implantable medical device and a constraining mechanism configured to constrain the implantable medical device to a delivery configuration. The constraining mechanism may include a single fiber arranged about the implantable medical device having a plurality of knots to maintain the constraining mechanism in constrained configuration with at least two of the plurality of knots being in contact in the constrained configuration.

### FIELD

The present disclosure relates to apparatuses, systems, and methods that include coverings for delivery of implantable medical devices. More specifically, the present disclosure relates to apparatuses, systems, and methods that include coverings for constraining an expandable device during device delivery.

### BACKGROUND

Stents and stent-grafts may be utilized to radially support a variety of tubular passages in the body, including arteries, veins, airways, gastrointestinal tracts, and biliary tracts. The preferred method of placing these devices has been to use specialized delivery systems to precisely place and deploy a device at the site to be treated. These delivery systems allow the practitioner to minimize the trauma and technical difficulties associated with device placements. Attributes of delivery systems include: low profile; ability to pass through introducer sheaths; ability to negotiate tortuous vasculature, smoothly and atraumatically; protection of constrained devices; and ability to accurately position and deploy the device.

Stents or stent-grafts may be deployed and plastically deform by using an inflatable balloon (e.g., balloon expandable stents) or to self-expand and elastically recover (e.g., “self expandable” stents) from a collapsed or constrained delivery diameter to an expanded and deployed diameter. Some stents are designed to elastically recover by being manufactured at their functional diameter of a material that has elastic recovery properties, and then radially compressed to be mounted on a delivery catheter.

These stent and stent-graft devices may be held, compressed, or constrained in the delivery configuration prior to and during delivery to a target location. The devices may be held in this compressed state for a prolonged period of time (e.g., after manufacture and prior to use). Different mechanisms or devices may be used to hold the stent and stent-graft devices in a delivery state and be removed to allow expansion of the stent and stent-graft devices at the target location.

### SUMMARY

According to one example (“Example 1”), a delivery system includes an implantable medical device; and a constraining mechanism configured to constrain the implantable medical device to a delivery configuration, the constraining mechanism including a single fiber arranged about the implantable medical device having a plurality of knots to maintain the constraining mechanism in constrained configuration with at least two of the plurality of knots being in contact in the constrained configuration.

According to another example (“Example 2”), further to Example 1, each of the plurality of knots are in contact with adjacent ones of the plurality of knots when the constraining mechanism is in the constrained configuration.

According to another example (“Example 3”), further to any one of Examples 1-2, the single fiber is configured to sequentially untie the plurality of knots in response to applied tension and release the constraining mechanism to allow expansion of the implantable medical device to a deployed configuration.

According to another example (“Example 4”), further to Example 3, the implantable medical device includes a stent having a plurality of apices, and the single fiber is configured to release in sequence and avoid catching on the apices during release.

According to another example (“Example 5”), further to Example 4, the plurality of knots are configured to maintain position relative to the implantable medical device in the constrained configuration prior to being released in sequence.

According to another example (“Example 6”), further to any one of Examples 4-5, the plurality of knots are configured to lessen ramping of the implantable medical device prior to being released in sequence.

According to another example (“Example 7”), further to any one of Examples 1-6, the plurality of knots are longitudinally aligned a longitudinal axis of the constraining mechanism.

According to another example (“Example 8”), further to any one of Examples 1-6, the plurality of knots alternate sides of a longitudinal axis of the constraining mechanism.

According to another example (“Example 9”), further to Example 8, the single fiber forms multiple loops are angled relative to the longitudinal axis of the constraining mechanism.

According to another example (“Example 10”), further to any one of Examples 1-8, the single fiber forms multiple loops arranged circumferentially about the implantable medical device and the multiple loops are packed at a density such that at least two loops of the multiple loops are in physical contact.

According to another example (“Example 11”), further to Example 10, the multiple loops are substantially perpendicular to a longitudinal axis of the constraining mechanism formed by the plurality of knots.

According to another example (“Example 12”), further to any one of Examples 10-11, the multiple loops are packed at a density with each loop being in physical contact with adjacent ones of the multiple loops.

According to another example (“Example 13”), further to any one of Examples 10-12, the multiple loops are packed at a density configured to substantially gaplessly cover the implantable medical device and the density is between approximately 0.006 inches and 0.1 inches.

According to another example (“Example 14”), further to Example 13, the implantable medical device comprises a drug eluting coating, and the multiple loops of the constraining mechanism are configured to lessen release of the drug eluting coating prior to the constraining mechanism releasing to allow expansion of the implantable medical device to a deployed configuration.

According to one example (“Example 15”), a method of removing a constraining mechanism with the method including arranging a medical device within the constraining mechanism in a constrained configuration, the constraining mechanism including a single fiber arranged about the medical device having a plurality of knots; and applying tension to an end of the single fiber to sequentially release the plurality of knots to allow release of the medical device to a deployed configuration from the constrained configuration.

According to another example (“Example 16”), further to Example 15, the implantable medical device includes a stent having a plurality of apices, releasing the plurality of knots in sequence avoids the single fiber catching on the apices during release.

According to another example (“Example 17”), further to any one of Examples 15-16, releasing the plurality of knots in sequence includes the plurality of knots maintaining position relative to the implantable medical device prior to being released in sequence.

According to one example (“Example 18”), an apparatus includes a constraining mechanism configured to constrain an implantable medical device, the constraining mechanism having a plurality of knots configure to release in sequence and multiple loops arranged circumferentially about the implantable medical device in a constrained configuration and a single fiber having a substantially unknotted structure in a non-constrained configuration.

According to another example (“Example 19”), further to Example 18, the plurality of knots are longitudinally aligned a longitudinal axis of the constraining mechanism and the multiple loops are substantially perpendicular to the longitudinal axis of the constraining mechanism

According to another example (“Example 20”), further to Example 18, the plurality of knots alternate sides of a longitudinal axis of the constraining mechanism.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is a top plan view of a catheter with a constraining mechanism, according to some embodiments.

FIG. 2 is an illustration of an example constraining mechanism, according to some embodiments.

FIG. 3 is an illustration of an example constraining mechanism and implantable medical device, according to some embodiments.

FIG. 4 is an illustration of another example constraining mechanism and implantable medical device, according to some embodiments.

The accompanying drawings are included to provide a further understanding of the disclosure and are incorporated in and constitute a part of this specification, illustrate embodiments, and together with the description serve to explain the principles of the disclosure.

### DETAILED DESCRIPTION

Persons skilled in the art will readily appreciate that various aspects of the present disclosure can be realized by any number of methods and apparatus configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

Various aspects of the present disclosure are directed toward apparatuses, methods, and systems that include a constraining mechanism configured to hold, compress, or constrain an implantable medical device (e.g., a stent or stent-graft) in a delivery configuration prior to and during delivery to a target location. In certain instances, the constraining mechanism includes a single fiber. The single fiber, as compared to certain sheaths, sleeves or multiple fiber constraining mechanisms, may constrain an implantable medical device at a smaller profile.

The single fiber, in certain instances, wraps the device circumferentially with each circumferential wrap of the single fiber being secured with a loop. The loop may include a loop knitting pattern along the length of the device with a plurality of knots. In addition, the single fiber constraining mechanism may facilitate deployment of the implantable medical device by avoiding catching on the implantable medical device and avoiding undesired pre-deployment of the device as discussed in further detail below. In particular, compared multiple fiber constraining mechanisms, the single fiber constraining mechanism can lessen the opportunity for catching and pre-deployment on the device.

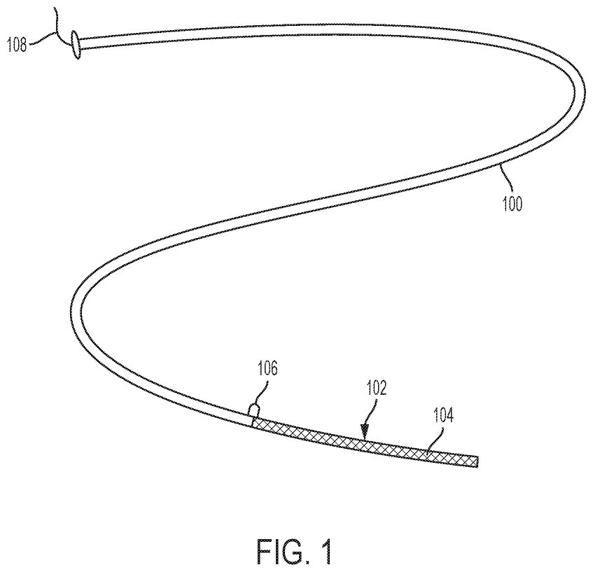

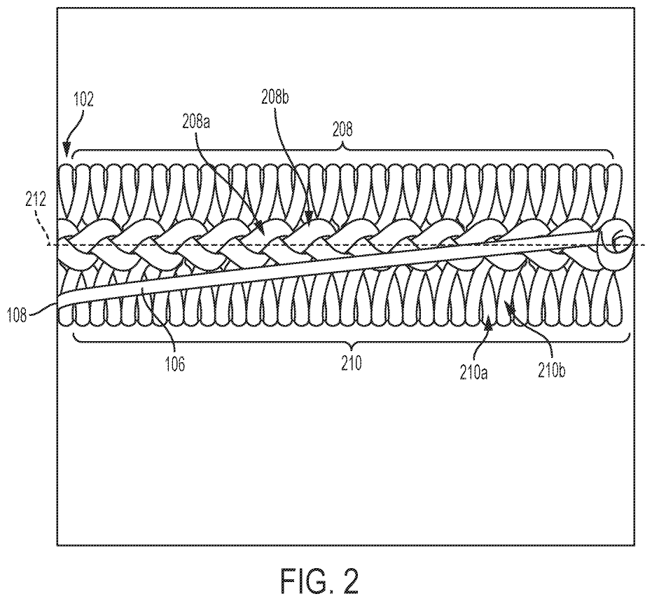

FIG. 1 is a top plan view of a catheter 100 with a constraining mechanism 102, according to some embodiments. As shown in FIG. 1, the constraining mechanism 102 is configured to constrain an implantable medical device 104 to a delivery configuration. The constraining mechanism 102 may include a single fiber 106 arranged about the implantable medical device 104 having a plurality of knots to maintain the constraining mechanism 102 in a constrained configuration. The single fiber 106 of the constraining mechanism 102, as shown in further detail with reference to FIGS. 2-4, may include a series of knots.

The constraining mechanism 102 is arranged along a length of the implantable medical device 104. The constraining mechanism 102 is also circumferentially arranged about the implantable medical device 104 and may substantially cover the implantable medical device 104 for delivery. In addition, and as shown in FIG. 1, the single fiber 106 may include a series of knots and is also circumferentially arranged about the implantable medical device 104 over the length of the implantable medical device 104 with the single fiber 106 being not knotted or otherwise wrapped proximal to the implantable medical device 104. The single fiber 106 may be arranged within a lumen (not shown) of the catheter 100 and extend toward a proximal end of the catheter 100 that is arranged external to a patient during delivery of the implantable medical device 104. The single fiber 106 includes a proximal end 108 that a user may apply tension to in order to release the constraining mechanism 102 and deploy the implantable medical device 104.

In certain instances, the single fiber 106 releases similar to a rip cord such that the knots sequentially release along the length of the implantable medical device 104. As is explained in greater detail below, the constraining mechanism 102 is formed by knitting together the single fiber 106 directly on the implantable medical device 104. As contrasted to prior multiple fiber constraining mechanisms which are knotted together and then subsequently arranged about a constrained device, the constraining mechanism 102 is formed directly on the implantable medical device 104 according to various examples. The implantable medical device 104 may be a stent, stent-graft, a balloon, or a similar device.

FIG. 2 is an illustration of an example constraining mechanism 102, according to some embodiments. The constraining mechanism 102 may be included a portion of a delivery system (e.g., a catheter and implantable medical device as shown in FIG. 1). FIG. 2 shows the constraining mechanism 102 in a constrained configuration in which an implantable medical device (not shown) is held to a diameter less than a deployed, expanded or working diameter. The constraining mechanism 102 is configured to constrain an implantable medical device to a delivery configuration. In addition, the constraining mechanism 102 includes a single fiber 106 arranged having a plurality of knots 208 to maintain the constraining mechanism 102 in a constrained configuration. The plurality of knots 208 are configured and arranged such that at least two of the plurality of knots 208 are in contact in the constrained configuration as shown in FIG. 2.

As highlighted in FIG. 2, adjacent knots 208a-b of the plurality of knots 208 are in physical contact. The knots 208a-b being in contact increases the density of the constraining mechanism 102 as compared to prior multi-fiber constrain devices. In certain instances, each of the plurality of knots 208 are in contact with adjacent knots as shown in FIG. 2. In addition, and in certain instances, the plurality of knots 208 are longitudinally aligned along longitudinal axis 212 of the constraining mechanism 102.

The alignment of the plurality of knots 208 can facilitate densely packing of the plurality of knots 208. In certain instances, the alignment of the plurality of knots 208 and at least two adjacent knots 208a-b of the plurality of knots 208 being in physical contact relates to an amount of force applied by the constraining mechanism 102. In certain instances, the fiber 106 may be wrapped around the implantable medical device at tension in a range between 300 g and over 1 kg.

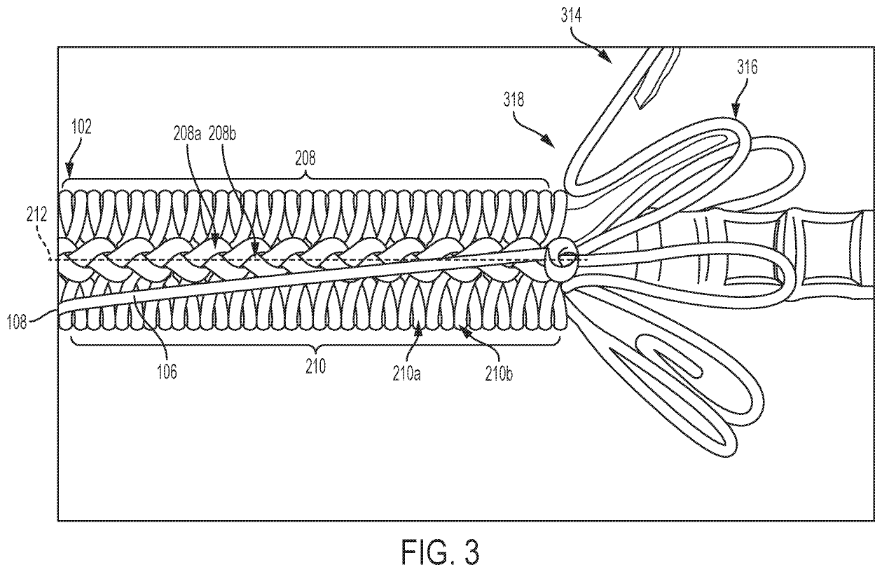

In addition, and as discussed in further detail with reference to FIG. 3, the alignment of the plurality of knots 208 and at least two adjacent knots 208a-b of the plurality of knots 208 being in physical contact facilitates deployment of an implantable medical device by avoiding catching on the implantable medical device and avoiding undesired pre-deployment of the device as discussed in further detail below.

In certain instances, the single fiber 106 forms multiple loops 210 arranged circumferentially about the implantable medical device. In addition, and as shown in FIG. 2, the multiple loops 210 are packed at a density such that at least two loops 210a-b of the multiple loops 210 are in physical contact. The loops 210 are unknotted portions of the single fiber 102 between knots of the plurality of knots 208. The multiple loops 210 may be arranged circumferentially about an implantable medical device and in certain instances, the multiple loops 210 are substantially perpendicular to the longitudinal axis 212 of the constraining mechanism 102.

As compared to prior multi-fiber constraining devices, the single fiber 106 is configured to prevent non-sequential tensioning and un-tensioning (e.g., releasing) that can complicate deployment. The plurality of knots 208 of the single fiber 106 forming the constraining mechanism 102 are an interlocking structure that unravels as a coherent interwoven rip cord by unknotting the plurality of knots 208 on the single fiber 106. As tension is applied to the proximal end 108 of the single fiber 106, the plurality of knots 208 release in sequence. This process will continue along the entire length of the device until each of the plurality of knots 208 disengage as one long, continuous, un-knotted single fiber 106.

FIG. 3 is an illustration of an example constraining mechanism 102 and implantable medical device 314, according to some embodiments. The constraining mechanism 102 is formed of a single fiber 106 that includes a plurality of knots 208 arranged along a longitudinal axis 212 of the constraining mechanism 102. In addition, the single fiber 106 forms multiple loops 210 arranged perpendicular to the longitudinal axis 212 of the constraining mechanism. The multiple loops 210 are also arranged circumferentially about the implantable medical device 314. The single fiber 106 includes a proximal end 108 that a user may apply tension to in order to release the constraining mechanism 102 and deploy the implantable medical device 104.

In certain instances, at least two adjacent knots 208a-b of the plurality of knots 208 are in physical contact and/or at least two loops 210a-b of the multiple loops 210 are in physical contact. The single fiber 106 may be configured to sequentially untie the plurality of knots 208 in response to applied tension and release the constraining mechanism 102 to allow expansion of the implantable medical device 314 to a deployed configuration.

In FIG. 3, the implantable medical device 314 is shown in a partially deployed configuration with the constraining mechanism 102 having been partially released. The implantable medical device 314 may be a stent that includes multiple apices 316 with a single apex highlighted in FIG. 3 for ease of illustration. As noted above, the single fiber 106 facilitates deployment by avoiding catching on the implantable medical device 314. Releasing the plurality of knots 208 in sequence avoid being caught on the apices 316 during release. The plurality of knots 208 may be released along the longitudinal axis 212 to avoid snagging on the apices 316. The single fiber 106 avoids shifting axially relative to the implantable medical device 314. In addition, releasing the plurality of knots 208 in sequence maintains a consistent deployment force during deployment of the expandable medical 314, which may avoid misdeployment or shifting of the constraining mechanism 102.

In certain instances, the plurality of knots 208 are configured to maintain position relative to the implantable medical device 314 in the constrained configuration prior to being released in sequence. The single fiber 106 may be configured to lessen ramping of the implantable medical device 314 prior to the plurality of knots 208 being released in sequence. As shown in FIG. 3, the expandable medical 314 begins to expand to a larger diameter after release of the constraining mechanism 102. The expandable medical 314 may be have an angle 318 between the portions held by the constraining mechanism 102 and portions that have been expanded or are beginning to expand. Due to the angle 318 and the expandable device 314 expending a force to deploy to the deployed diameter, prior devices may shift due to ramping of the implantable medical device 314. The axial shifting of the constraining mechanism 102 could result in pre-deployment or the constraint to get caught on apices of the implantable medical device 314. The single fiber 106, however, is able to lessen ramping of the implantable medical device 314 by maintaining a location of each of the plurality of knots 208, relative to the implantable medical device 314, as the plurality of knots 208 are released in sequence. The single fiber 106, in this manner, lessens undesired or pre-deployment of the implantable medical device 314.

The multiple loops 210 of the constraining mechanism 102 may be packed at a density such that each loop 210 is in physical contact with adjacent ones 210a-c of the multiple loops 210 as noted above. In certain instances, the multiple loops 210 are packed at a density configured to substantially gaplessly cover the implantable medical device 314. In certain instances, the density is between approximately 0.006 inches and 0.1 inches. Further, the implantable medical device 314 may include a drug eluting coating and the multiple loops 210 of the constraining mechanism 102 are configured to lessen release of the drug eluting coating prior to the constraining mechanism 102 releasing to allow expansion of the implantable medical device 314 to a deployed configuration.

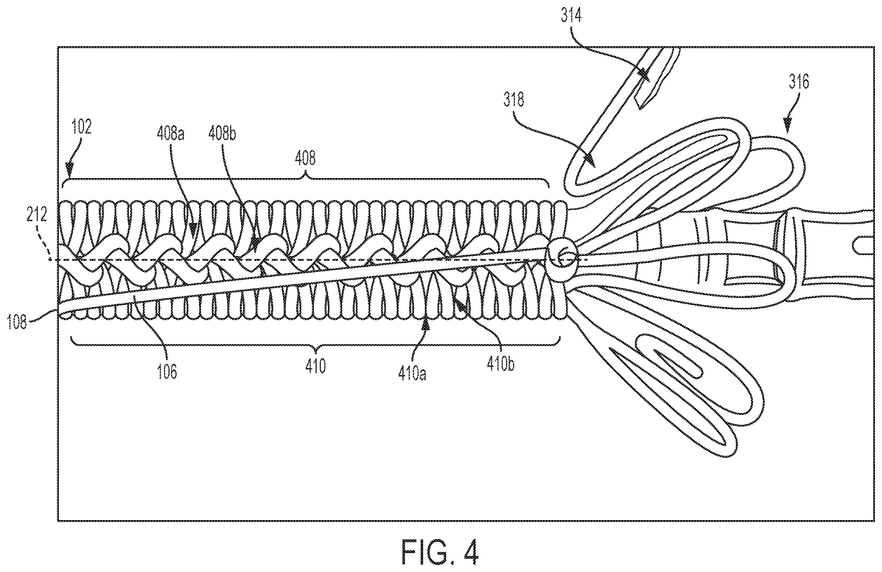

FIG. 4 is an illustration of another example constraining mechanism 102 and implantable medical device 314, according to some embodiments. As discussed in further detail above with reference to FIGS. 2-3, the constraining mechanism 102 is formed of a single fiber 106 that includes a plurality of knots 408. In addition, the single fiber 106 forms multiple loops 410 arranged about a circumference of the constraining mechanism 102 between the plurality of knots 408.

As shown in FIG. 4, the plurality of knots 408 are arranged on alternating sides of a longitudinal axis 212 of the constraining mechanism 102. In addition, the multiple loops 410 are also arranged circumferentially about the implantable medical device 314. The single fiber 106 forms the multiple loops 410 in an angled configuration circumferentially about the implantable medical device 314. The multiple loops 410 may be angled relative to the longitudinal axis 21 of the constraining mechanism 102. In certain instances, at least two adjacent knots 408a-b of the plurality of knots 408 are in physical contact. Further, at least two loops 410a-b of the multiple loops 410 are in physical contact. The single fiber 106 may be configured to sequentially untie the plurality of knots 408 in response to applied tension and release the constraining mechanism 102 to allow expansion of the implantable medical device 314 to a deployed configuration.

In FIG. 4, the implantable medical device 314 is shown in a partially deployed configuration with the constraining mechanism 102 being partially released. In certain instances, the plurality of knots 408 are configured to maintain position relative to the implantable medical device 314 in the constrained configuration prior to being released in sequence. As shown in FIG. 4, the implantable medical device 314 begins to expand to a larger diameter after release of the constraining mechanism 102. The implantable medical device 314 may be have an angle 318 between the portions held by the constraining mechanism 102 and portions that have been expanded or are beginning to expand. Due to the angle 318 and the expandable device 314 exerts a force to deploy to the deployed diameter, prior devices may shift due to ramping of the implantable medical device 314. The single fiber 106, however, lessens ramping of the implantable medical device 314 by maintaining a location of each of the plurality of knots 408, relative to the implantable medical device 314, as the plurality of knots 408 are released in sequence. The single fiber 106, in this manner, lessens undesired or pre-deployment of the implantable medical device 314.

The device shown in FIG. 4 is provided as an example of the various features of the constraining mechanism 102 and, although the combination of those illustrated features is clearly within the scope of invention, that example and its illustration is not meant to suggest the inventive concepts provided herein are limited from fewer features, additional features, or alternative features to one or more of those features shown in FIG. 4. For example, in various embodiments, the constraining mechanism 102 shown in FIG. 4 may include the density of loops described with reference to FIG. 3. It should also be understood that the reverse is true as well. One or more of the components depicted in FIG. 4 can be employed in addition to, or as an alternative to components depicted in FIGS. 2-3. For example, the alternating knots 408 of the constraining mechanism 102 shown in FIG. 4 may be employed in connection with the constraining mechanism 102 of FIG. 2-3.

The materials used to make the single fiber 106 of the present invention are likewise open to modification and customization for given applications. For most uses discussed herein the single fiber 106 used to form the constraining mechanism 102 may include: polytetrafluoroethylene (PTFE); expanded PTFE; silk; thermoplastic threads such as polypropylene; polyamide (nylon); various plastic or metal materials (e.g., stainless steel or nickel-titanium (nitinol) alloy); and bioresorbable materials, such as PLA or PGA. Particularly preferred for use in covering implantable medical devices are polytetrafluoroethylene (PTFE) threads, and especially expanded PTFE threads, such as threads available from W. L. Gore & Associates, Inc., Elkton, Md., under the trademark RASTEX® or sutures available from W. L. Gore & Associates, Inc., Flagstaff, Ariz., under the trademark GORE-TEX®.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. A delivery system comprising:
an implantable medical device; and
a constraining mechanism configured to constrain the implantable medical device to a delivery configuration, the constraining mechanism including a single fiber arranged about the implantable medical device having a plurality of knots to maintain the constraining mechanism in constrained configuration with at least two of the plurality of knots being in contact in the constrained configuration.

2. The system of claim 1, wherein each of the plurality of knots are in contact with adjacent ones of the plurality of knots when the constraining mechanism is in the constrained configuration.

3. The system of claim 1, wherein the single fiber is configured to sequentially untie the plurality of knots in response to applied tension and release the constraining mechanism to allow expansion of the implantable medical device to a deployed configuration.

4. The system of claim 3, wherein the implantable medical device includes a stent having a plurality of apices, and the single fiber is configured to release in sequence and avoid catching on the apices during release.

5. The system of claim 4, wherein the plurality of knots are configured to maintain position relative to the implantable medical device in the constrained configuration prior to being released in sequence.

6. The system of claim 4, wherein the plurality of knots are configured to lessen ramping of the implantable medical device prior to being released in sequence.

7. The system of claim 1, wherein the plurality of knots are longitudinally aligned a longitudinal axis of the constraining mechanism.

8. The system of claim 1, wherein the plurality of knots alternate sides of a longitudinal axis of the constraining mechanism.

9. The system of claim 8, wherein the single fiber forms multiple loops are angled relative to the longitudinal axis of the constraining mechanism.

10. The system of claim 1, wherein the single fiber forms multiple loops arranged circumferentially about the implantable medical device and the multiple loops are packed at a density such that at least two loops of the multiple loops are in physical contact.

11. The system of claim 10, wherein the multiple loops are substantially perpendicular to a longitudinal axis of the constraining mechanism formed by the plurality of knots.

12. The system of claim 10, wherein the multiple loops are packed at a density with each loop being in physical contact with adjacent ones of the multiple loops.

13. The system of claim 10, wherein the multiple loops are packed at a density configured to substantially gaplessly cover the implantable medical device and the density is between approximately 0.006 inches and 0.1 inches.

14. The system of claim 13, wherein the implantable medical device comprises a drug eluting coating, and the multiple loops of the constraining mechanism are configured to lessen release of the drug eluting coating prior to the constraining mechanism releasing to allow expansion of the implantable medical device to a deployed configuration.

15. A method of removing a constraining mechanism, the method comprising:
arranging a medical device within the constraining mechanism in a constrained configuration, the constraining mechanism including a single fiber arranged about the medical device having a plurality of knots; and
applying tension to an end of the single fiber to sequentially release the plurality of knots to allow release of the medical device to a deployed configuration from the constrained configuration.

16. The method of claim 15, wherein the implantable medical device includes a stent having a plurality of apices, releasing the plurality of knots in sequence avoids the single fiber catching on the apices during release.

17. The method of claim 15, wherein releasing the plurality of knots in sequence includes the plurality of knots maintaining position relative to the implantable medical device prior to being released in sequence.

18. An apparatus comprising:
a constraining mechanism configured to constrain an implantable medical device, the constraining mechanism including:
a plurality of knots configure to release in sequence and multiple loops arranged circumferentially about the implantable medical device in a constrained configuration, and
a single fiber having a substantially unknotted structure in a non-constrained configuration.

19. The apparatus of claim 18, wherein the plurality of knots are longitudinally aligned a longitudinal axis of the constraining mechanism and the multiple loops are substantially perpendicular to the longitudinal axis of the constraining mechanism

20. The apparatus of claim 18, wherein the plurality of knots alternate sides of a longitudinal axis of the constraining mechanism.
